(19) United States
(12) Patent Application Publication
(10) Pub. No.: US 2024/0260844 A1
(43) Pub. Date: Aug. 8, 2024

(54) VASCULAR STATE MEASURING DEVICE

(71) Applicant: NATIONAL TSING HUA UNIVERSITY, Hsinchu City (TW)

(72) Inventor: Ting-Wei WANG, Hsinchu City (TW)

(73) Assignee: NATIONAL TSING HUA UNIVERSITY, Hsinchu City (TW)

(21) Appl. No.: 18/462,165

(22) Filed: Sep. 6, 2023

(30) Foreign Application Priority Data
Feb. 3, 2023 (TW) 112103912

(51) Int. Cl. A61B 5/021; A61B 5/00; A61B 5/05

(52) U.S. Cl. CPC A61B 5/021; A61B 5/05; A61B 5/6826

(57) ABSTRACT

A vascular state measuring device includes a signal transceiver and a control module. The signal transceiver has a tunnel structure for arranging a to-be-measured part. The signal transceiver is configured to output an electromagnetic signal to the to-be-measured part towards the tunnel structure to generate an eddy current at the to-be-measured part, and receive an electromagnetic signal generated by the corresponding eddy current. The control module is coupled with the signal transceiver, and the control module includes a signal generation unit and a processing unit. The signal generation unit is configured to generate an AC signal and provide the AC signal to the signal transceiver to generate the first electromagnetic signal. The processing unit is configured to calculate a characteristic signal based on the first electromagnetic signal and the second electromagnetic signal. The characteristic signal corresponds to at least one state of at least one blood vessel at the to-be-measured part.

### TECHNICAL FIELD

The present invention relates to a vascular state measuring device, and in particular, to a wearable non-invasive vascular state measuring device with a magnetoelectric effect sensing mechanism.

### BACKGROUND

At present, most wearable products for measuring heart rate and/or blood pressure on the market use photoplethysmography (PPG). However, the measurement mechanism may affect the accuracy and value of heart rate and/or blood pressure measurement due to the change in the measurement position caused by incorrect wearing. For example, an excessively loose watch band of a wristwatch-type measuring device may lead to the sliding of the measuring device, causing poor transmission or reception of a light-emitting elements and a light receiver in the measuring device, and further affecting the detection of heart rate and/or blood pressure. Furthermore, PPG uses the physical principle of optical transmission and reflection. Due to the physical limitation of optics, different optical transmission and reflection efficiencies may be caused due to different skin colors and/or skin keratin thicknesses of different subjects, which may further affect the accuracy of measurement.

Therefore, when pursuing portability or convenience of a device, people also focus on technical development in the art to avoid measurement errors caused by individual differences in users.

### SUMMARY

An object of the present invention is to provide a non-invasive vascular state measuring device that can be stably worn.

An object of the present invention is to provide a non-invasive vascular state measuring device for avoiding measurement errors caused by individual differences in users.

The present invention provides a vascular state measuring device comprising a signal transceiver and a control module. The signal transceiver has a tunnel structure for arranging a to-be-measured part. The signal transceiver is configured to output at least a first electromagnetic signal to the to-be-measured part towards the tunnel structure to generate an eddy current at the to-be-measured part, and receive a second electromagnetic signal generated by the corresponding eddy current. The control module is coupled with the signal transceiver, and the control module includes a signal generation unit and a processing unit. The signal generation unit is configured to generate an AC signal and provide the AC signal to the signal transceiver to generate the first electromagnetic signal. The processing unit is configured to calculate a characteristic signal based on the first electromagnetic signal and the second electromagnetic signal. The characteristic signal corresponds to at least one state of at least one blood vessel at the to-be-measured part.

As described above, the signal transceiver of the present vascular state measuring device has a tunnel structure, so that, a finger or a wrist of a subject can pass through and the to-be-measured part may be arranged in the tunnel structure. The tunnel structure has a relatively stable structure to avoid improper operation during wearing. The tunnel structure can also improve the convenience and stability during wearing the vascular state measuring device. The presented vascular state measuring device measures the at least one state of the at least one blood vessel at the to-be-measured part by an eddy current mechanism, which will avoid measurement errors caused by individual differences in subjects or differences in measured parts.

### BRIEF DESCRIPTION OF THE DRAWINGS

The accompanying drawings are presented to help describe various aspects of the present invention. In order to simplify the accompanying drawings and highlight the contents to be presented in the accompanying drawings, conventional structures or elements in the accompanying drawings may be drawn in a simple schematic way or may be omitted. For example, a number of elements may be singular or plural. These accompanying drawings are provided merely to explain these aspects and not to limit them.

FIG. 1 is a schematic diagram of a vascular state measuring device according to a first embodiment of the present invention.

FIG. 2 is a schematic cross-sectional view of the vascular state measuring device after being worn according to a first embodiment of the present invention.

FIG. 3 is a schematic diagram of a vascular state measuring device according to a second embodiment of the present invention.

FIG. 4 is a schematic cross-sectional view of the vascular state measuring device after being worn according to a second embodiment of the present invention.

FIG. 5 is a schematic diagram of a vascular state measuring device with a matching element according to a third embodiment of the present invention.

FIG. 6 is a schematic diagram of a vascular state measuring device including a depth detection unit according to a fourth embodiment of the present invention.

FIG. 7 is a schematic diagram of a vascular state measuring device scanning a leading electromagnetic signal according to a fifth embodiment of the present invention.

FIG. 8 is a schematic diagram of a vascular state measuring device including an adjustable passive element according to a fifth embodiment of the present invention.

FIG. 9 is a schematic diagram of transmission between a vascular state measuring device and an electronic device according to a sixth embodiment of the present invention.

### DETAILED DESCRIPTION

Any reference to elements using names such as “first” and “second” herein generally does not limit the number or order of these elements. Conversely, these names are used herein as a convenient way to distinguish two or more elements or element instances. Therefore, it should be understood that the names “first” and “second” in the request item do not necessarily correspond to the same names in the written description. Furthermore, it should be understood that references to the first element and the second element do not indicate that only two elements can be used or that the first element needs to precede the second element. Open terms such as “include”, “comprise”, “have”, “contain”, and the like used herein means including but not limited to.

The term “coupled” is used herein to refer to direct or indirect electrical coupling between two structures. For example, in an example of indirect electrical coupling, one structure may be coupled with another structure through a passive element such as a resistor, a capacitor, or an inductor.

In the present invention, the term such as “exemplary” or “for example” is used to represent “giving an example, instance, or description”. Any implementation or aspect described herein as “exemplary” or “for example” is not necessarily to be construed as preferred or advantageous over other aspects of the present invention. The terms “about” and “approximately” as used herein with respect to a specified value or characteristic are intended to represent within a value (for example, 10%) of the specified value or characteristic.

In the present invention, the “vascular state” referred to herein is, for example, but not limited to, parameters of medical or non-medical significance such as vasoconstriction and/or dilation, pulse, blood vessel elasticity, an intravascular status (for example, whether inside of the blood vessel is blocked or unblocked, a blood flow state, and a blood flow velocity), blood vessel hyperplasia, a blood vessel density, and a blood vessel wall state (for example, whether the blood vessel wall is damaged).

### First Embodiment

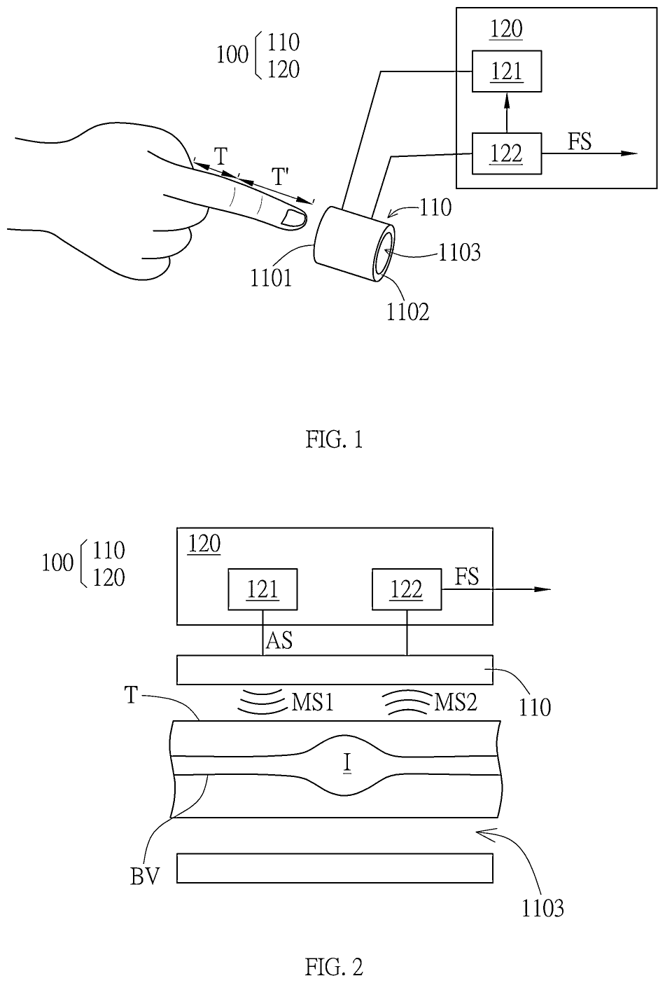

Referring to FIG. 1 to FIG. 2, the vascular state measuring device 100 is provided. The vascular state measuring device 100 comprises the signal transceiver 110 and the control module 120. The signal transceiver 110 has the tunnel structure 1103 for arranging the to-be-measured part T. The signal transceiver 110 is configured to output the first electromagnetic signal MS1 to the to-be-measured part T towards the tunnel structure to generate the eddy current I at the to-be-measured part T, and configured to receive the second electromagnetic signal MS2 generated by the eddy current I induced by the first electromagnetic signal MS1 at the to-be-measured part T. The control module 120 is coupled with the signal transceiver 110. The control module 120 includes the signal generation unit 121 and the processing unit 122. The signal generation unit 121 is configured to generate an AC signal AS and provide the AC signal to the signal transceiver 110 to generate the first electromagnetic signal MS1. The processing unit 122 is configured to calculate the characteristic signal FS based on the first electromagnetic signal MS1 and the second electromagnetic signal MS2. The characteristic signal FS corresponds to at least one state of at least one blood vessel BV at the to-be-measured part T.

The signal transceiver 110 has the tunnel structure 1103. Specifically, referring to FIG. 1, the through hole 1103 is provided between the first end surface 1101 and the second end surface 1102 of the signal transceiver 110. The front end part T′ of the to-be-measured part T may extend through the through hole 1103, so that the to-be-measured part T can be arranged in the through hole (tunnel structure) 1103. For example, when the to-be-measured part T is between the first knuckle and the second knuckle of the finger, the front end part T′ is the part from the fingertip to the second knuckle. On the other hand, the signal transceiver 110 may include the coil or another element with an electromagnetic wave transmitting/receiving function. An annular coil is used as an example. The hollow part of the coil serves as a part of the tunnel structure 1103. In other words, the front segment T′ of the finger of the subject may extend through the coil, so that the to-be-measured part T is arranged in the tunnel structure 1103. However, the to-be-measured part T may also be a fingertip, a wrist, an arm, a thigh, a calf, and the like. The to-be-measured part T of the present invention is not limited to the example in this embodiment. It should be noted that the cross section of the through hole 1103 is not necessarily circular, and the cross section of the through hole 1103 may be but not limited to circular, square, or polygonal. In addition, the signal transceiver 110 may be directly composed of coils, or the coils may be integrated into a housing or a soft cover, and the tunnel structure 1103 is composed of the housing or the soft cover.

The control module 120 is coupled with the signal transceiver 110. For example, the control module 120 may be an independent module coupled with the signal transceiver 110 in a wired/wireless manner. The control module 120 may also be integrated with the signal transceiver 110 in a housing or a soft cover to form an integrated electronic device. For example, the control module 120 may integrate the signal generation unit 121 and the processing unit 122 through a printed circuit board (PCB), a flexible circuit board (FPC), a glass substrate, and/or a silicon substrate. The signal generation unit 121 may be an AC/DC signal generation unit composed of active components (for example, an oscillator and a timer) and/or passive components (for example, a resistor, a capacitor, and an inductor). The processing unit 122 may be a unit with computing power composed of elements such as a microprocessor, a field programmable logic gate (FPGA), and an application specific integrated circuit (ASIC) with computing or programming capabilities and necessary active and passive elements (for example, an analog-digital conversion circuit, a capacitance meter, and an inductance meter).

Referring to FIG. 2, the signal generation unit 121 is configured to provide the AC signal AS to the signal transceiver 110, and the signal transceiver 110 generates the first electromagnetic signal MS1 due to the electromagnetic effect. The signal transceiver 110 is configured to output the first electromagnetic signal MS1 to the to-be-measured part T to generate an eddy current I at the to-be-measured part T. Specifically, after the first electromagnetic signal MS1 is applied to the to-be-measured part T, the to-be-measured part T (for example, the tissue, blood vessel, or blood at the to-be-measured part T may be regarded as a conducting plane) may generate the eddy current I correspondingly due to the first electromagnetic signal MS1. The eddy current I may generate a second electromagnetic signal MS2 in a direction opposite to a magnetic field direction of the first electromagnetic signal MS1. The second electromagnetic signal MS2 is to be received by the signal transceiver 110. In other words, the second electromagnetic signal MS2 generates a magnetoelectric effect on the signal transceiver 110 to generate an induced AC signal. The processing unit 122 is configured to measure the induced AC signal and generate the characteristic signal FS. For example, the characteristic signal FS may be a capacitance value, an inductance value, and/or an impedance value variation of the signal transceiver 110. In addition, the second electromagnetic signal MS2 or a difference between the first electromagnetic signal MS1 and the second electromagnetic signal MS2 (for example, a frequency variation and an amplitude variation) may also be directly measured as the characteristic signal FS. The characteristic signal FS may correspond to the blood vessel state of the blood vessel BV, for example. For example, a blood volume in the blood vessel BV increases and/or decreases based on contraction and relaxation of the blood vessel BV. Alternatively, the contraction and relaxation volume or a number of times per unit time is calculated to estimate parameters such as pulse or blood pressure.

Through the tunnel structure 1103 of the signal transceiver 110, the to-be-measured part T may be arranged in the tunnel structure 1103. The tunnel structure 1103 has a relatively stable structure, which can avoid improper operation during wearing, and can also improve the convenience and stability during wearing. Measurement by using the mechanism that the first electromagnetic signal MS1 generates the eddy current I may also avoid the measurement error caused by a difference in the to-be-measured parts T (for example, skin color and clothes).

### Second Embodiment

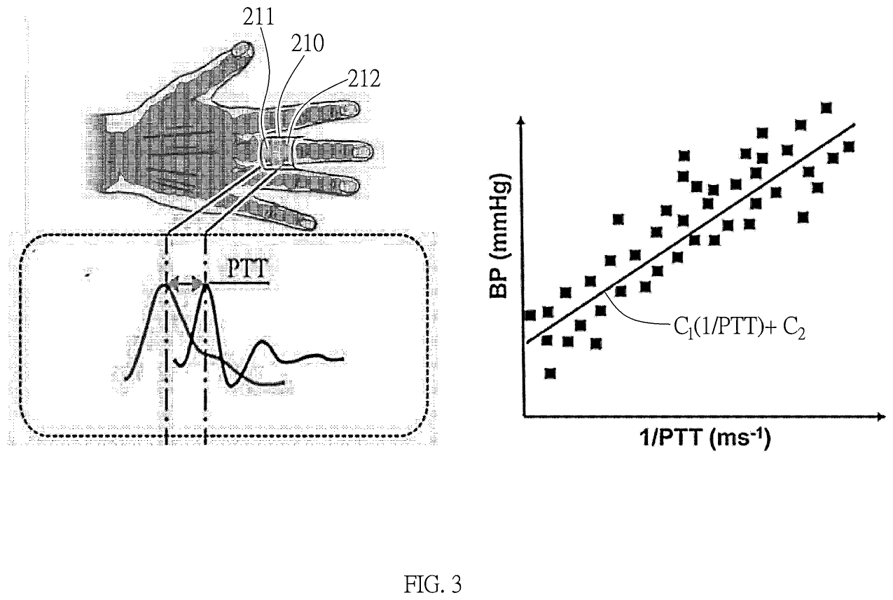

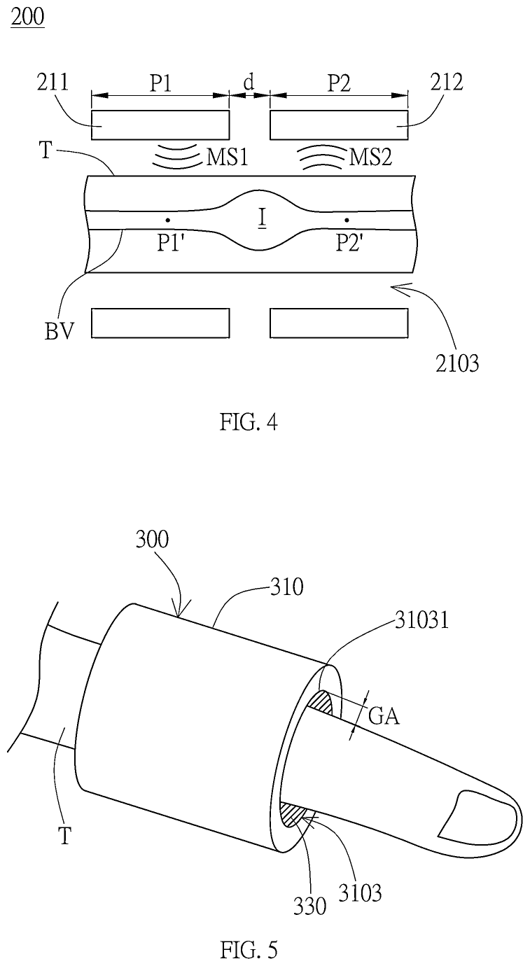

Referring to FIG. 3 to FIG. 4, in this embodiment, the signal transceiver 210 may further include the first coil 211 and the second coil 212. The first coil 211 and the second coil 212 are arranged at the first position P1 in the tunnel structure 2103. The second coil 212 is arranged at the second position P2 in the tunnel structure 2103, and hollow parts of the first coil 211 and the second coil 212 respectively serve as a part of the tunnel structure 2103. The spacing d is defined between the first position P1 and the second position P2. The time difference PPT exists between receiving of the second electromagnetic signal MS2 by the first coil 211 and receiving of the second electromagnetic signal by the second coil 212. The processing unit 222 calculates the blood vessel state of the blood vessel BV based on the spacing d and the time difference Td.

Specifically, the first coil 211 and the second coil 212 may respectively measure pulse propagation times (PPT) at two positions (P1′ and P2′) of the blood vessel BV. The pulse propagation time may be used to speculate on the blood vessel status between the two positions P1′ and P2′ of the blood vessel BV, for example, conditions such as blood vessel blockage and blood vessel rupture. On the other hand, in virtue of the Bramwell-Hill equation (such as Equation 1), it may be learned that the pulse propagation time PPT is negatively correlated with blood pressure.

$$d P = ρ {( \frac{D}{PTT} )}^{2} \frac{dA}{A} \qquad Equation 1$$

where dP represents a change in the blood vessel pressure, p represents the blood density, D is the spacing between positions P1′ and P2′, A is a basic value of the blood vessel cross-sectional area, and dA is a change in the blood vessel cross-sectional area. Equation 2 may be obtained after sorting equation 1 by using the cuff-base method.

$$B P = {C}_{1} \frac{1}{P T T} + {C}_{2} \qquad Equation 2$$

where BP is the blood vessel pressure, and C₁ and C₂ are respectively calibration parameters. As shown in the figure, the calibration parameters C₁ and C₂ may obtain regression curves by adding test data through big data or statistical methods. In this way, a linear equation with 1/PTT as a variable, the calibration parameter C₁ as a coefficient, and the calibration parameter C₂ as a constant may be deduced.

In this embodiment, in order to simplify the description, only the first coil 211 and the second coil 212 are used as examples. However, a person skilled in the art should know that the signal transceiver 210 may be provided with a plurality of coils. Through measurement with a plurality of coils, more time parameters may be obtained, or used in the remaining mathematical applications such as correction and difference, so as to achieve more accurate measurement results.

### Third Embodiment

In this embodiment, referring to FIG. 5, the vascular state measuring device 300 may further include the matching element 330. The matching element 330 is arranged on an inner wall 31031 of the tunnel structure 3103 of the signal transceiver 310. Specifically, the gap GA may exist between the to-be-measured part T and the inner wall 31031 of the tunnel structure 3103. The gap GA may cause, for example, the signal transceiver 310 to slip or misalign. In this embodiment, the matching element 330 may be made of an elastic or soft material to fill the gap GA, so as to avoid the measurement inaccuracy caused by the displacement of the signal transceiver 310. In terms of signal transmission, in this embodiment, the matching element 330 may be made of a material which has magnetic impedance in a range between the magnetic impedance of the to-be-measured part T and the magnetic impedance of the signal transceiver 310. Specifically, in this way, the energy loss during the energy transfer between the first electromagnetic signal MS1 and the second electromagnetic signal MS2 is reduced, so as to achieve the purpose of measuring the required signal or improving the signal-to-noise ratio with less energy, thereby avoiding problems such as injury to the subject or insufficient endurance of the device caused by excessive energy. However, the purpose of arranging the matching element 330 is not limited to the above example.

The matching element 330 may be used as a buffer arranged between the signal transceiver 310 and the to-be-measured part T. For example, the comfort of the subject or the stability during measurement can be improved. In terms of signal transmission, a proper dielectric material may be selected to improve the energy transfer efficiency of the first electromagnetic signal MS1 and the second electromagnetic signal MS2. The power consumption of the vascular state measuring device 300 can be reduced. Efficient energy transfer can also greatly reduce the risk of subjects being exposed to electromagnetic waves.

### Fourth Embodiment

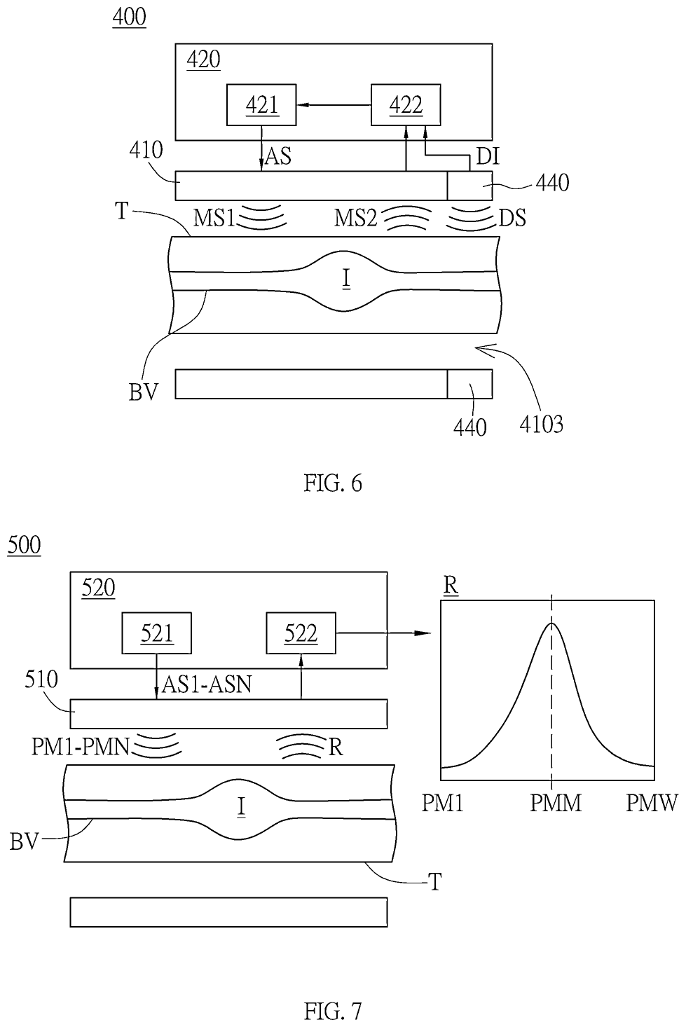

In this embodiment, referring to FIG. 6, the vascular state measuring device 400 further includes the depth detection unit 440. The depth detection unit 440 is configured to send the detection signal DS to the to-be-measured part T and provide depth information DI corresponding to the blood vessel BV to the control module 420. The control module 420 adjusts the frequency or the strength of the first electromagnetic signal MS1 based on the depth information DI. For example, the control module 420 may control the signal generation unit 421 to adjust the frequency or amplitude of the AC signal AS based on the depth information DI to generate the first electromagnetic signal MS1 with different frequencies and/or strengths. Alternatively, the first electromagnetic signal MS1 may be adjusted by adjusting the electrical characteristics (for example, an impedance value, an inductance value, and a capacitance value) of the signal transceiver 410. However, the manner of adjusting the first electromagnetic signal MS1 is not limited thereto. On the other hand, the depth detection unit 440 may be a unit composed of optical (the detection signal is an optical signal) or acoustic (the detection signal is an acoustic signal) elements of a detection mechanism with penetrability (for example, penetrating skin, cloth, or other media). The depth detection unit 440 measures a depth of a blood vessel in a target area through a ranging mechanism such as time-of-flight ranging (TOF). However, the elements and mechanisms for measuring the depth information DI are not limited thereto.

The depth detection unit 440 may be integrated with the signal transceiver 410 and the control module 420 in the housing. The depth detection unit 440 is preferably arranged at a position facing the tunnel structure 4103, so as to achieve a better effect of determining the depth. Through the depth information DI provided by the depth detection unit 440, the control module 420 may select a better signal for measurement (for example, through the processing unit 422), thereby improving the energy transfer efficiency of the first electromagnetic signal MS1 and the second electromagnetic signal MS2. The power consumption of the vascular state measuring device 400 can be reduced. Efficient energy transfer can also greatly reduce the risk of subjects being exposed to electromagnetic waves. On the other hand, based on the depth information DI, the first electromagnetic signal MS1 may also be focused on a target depth by using a focusing method such as a phase array. In this way, better measurement quality and a better signal-to-noise ratio are achieved.

### Fifth Embodiment

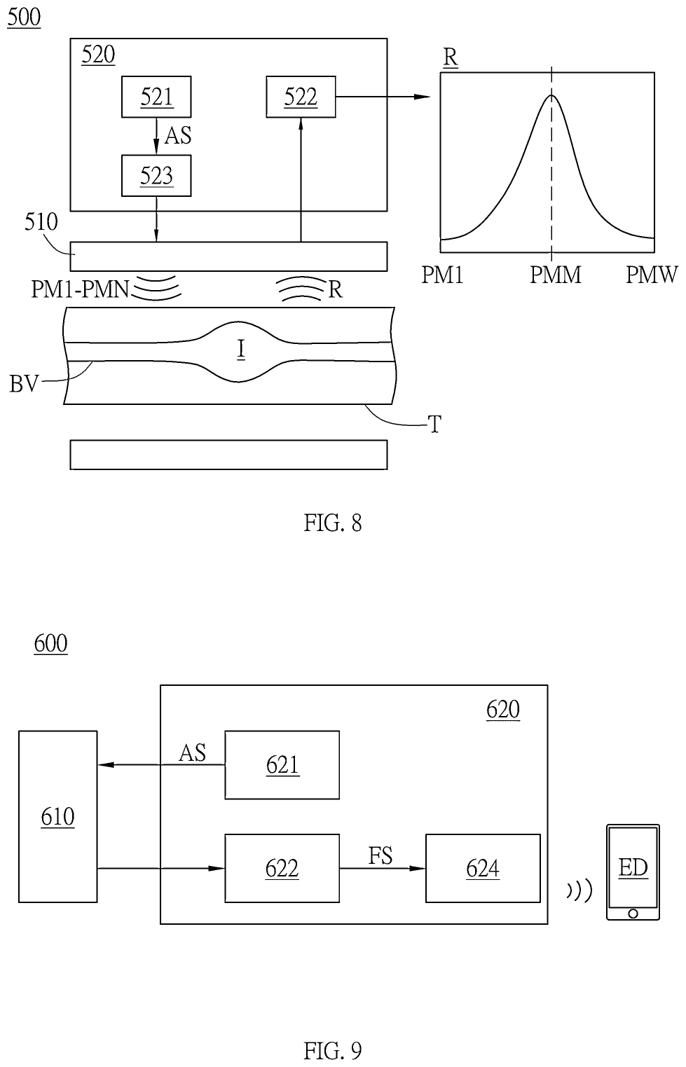

In this embodiment, as shown in FIG. 7 to FIG. 8, before outputting the first electromagnetic signal MS1, the signal transceiver 510 of the vascular state measuring device 500 further outputs at least one leading electromagnetic signal PMI-PMN. Each of the at least one leading electromagnetic signal PMI-PMN corresponds to a different signal parameter, and the signal parameter of the first electromagnetic signal MS1 corresponds to one (the PMM shown in FIG. 7) of the at least one leading electromagnetic signal PMI-PMN with an optimal response.

Specifically, the signal transceiver 510 generates the first electromagnetic signal MS1 based on the AC signal AS provided by the signal generation unit 521. However, different subjects or different to-be-measured parts may use different signal parameters (for example, a frequency, an amplitude, and a strength) to obtain optimal/better measurement results. Therefore, before the signal transceiver 510 outputs the first electromagnetic signal MS1, at least one leading electromagnetic signal PMI-PMN is used for pre-scanning, thereby selecting the best or relatively better first electromagnetic signal MS1 for measurement.

In this embodiment, as shown in FIG. 7, the signal generation unit 521 may be configured to output at least one leading AC signal AS1-ASN to generate the at least one leading electromagnetic signal PMI-PMN. Each of the at least one leading electromagnetic signal PMI-PMN corresponds to one of the at least one leading AC signal AS1-ASN. Specifically, the signal generation unit 521 outputs the leading AC signals AS1-ASN with different frequencies in a possible frequency interval (for example, kilohertz to megahertz) in sequence (for example, in ascending or descending order of the frequencies). Therefore, the leading electromagnetic signals PMI-PMN are generated, and the frequencies of the leading electromagnetic signals PMI-PMN are different corresponding to the frequencies of the leading AC signals AS1-ASN. By scanning the leading electromagnetic signals PMI-PMN with different frequencies, eddy currents I with different magnitudes may be generated at the to-be-measured part T, and corresponding responses with different magnitudes may be generated. For example, responsive electromagnetic signals of different strengths may be generated. The control module 520 (for example, through the processing unit 522) may determine a frequency of the AC signal AS outputted by the signal generation unit 521 based on a transmission frequency (PMM) corresponding to the largest or best one of the responses received by the signal transceiver 510, to generate the best or relatively better first electromagnetic signal MS1 for measurement.

In this embodiment, another mechanism for regulating signal parameters is shown in FIG. 8. The control module 520 includes an adjustable passive element 523 coupled with the signal transceiver 510, and the control module 520 adjusts a capacitance value, an inductance value, and/or an impedance value of the adjustable passive element 523 to adjust the signal parameter of each of at least one leading electromagnetic signal PMI-PMN. Specifically, the adjustable passive element 523 is coupled with the signal transceiver 510, and the adjustable passive element 523 may be adjusted by, for example, an adjustable capacitor. The capacitance value of the adjustable capacitor may indirectly adjust the capacitance value and/or impedance value of the signal transceiver 510. Therefore, when the leading electromagnetic signals PMI-PMN are to be generated, the signal parameters of each of the leading electromagnetic signals PMI-PMN may be different by changing the capacitance value of the adjustable passive element 523 through the AC signal AS provided by the signal generation unit 521. Eddy currents I with different magnitudes are generated at the to-be-measured part T through the leading electromagnetic signals PMI-PMN, and responses with different magnitudes are correspondingly generated. Therefore, the best/relatively better capacitor value can be selected. It should be noted that the adjustable passive element 523 is not limited to adjustment of the capacitance value, and the adjustable passive element 523 may be an element that is adjusted for at least one of the resistance value, the impedance value, the capacitance value, and/or the inductance value.

Through the leading electromagnetic signals PMI-PMN, the vascular state measuring device 500 may be calibrated before measurement, so that the measurement parameters most suitable for the current subject or the tested part can be generated. In this way, the measurement error caused by the subject or the tested part can be avoided. In addition, selection of preferable measurement parameters can also improve the measurement efficiency. The power consumption of that vascular state measuring device 500 can be reduced, and the risk of electromagnetic waves can be reduced.

### Sixth Embodiment

In this embodiment, as shown in FIG. 9, the vascular state measuring device 600 comprises the signal transceiver 610 and the control module 620. The control module 620 includes the signal generation unit 621, the processing unit 622, and the communication module 624 coupled with the processing unit 622 and configured to output the characteristic signal FS to an electronic device ED. Specifically, the electronic device ED is, for example, a back-end device such as a smart phone, a desktop computer, or a notebook computer. The communication module 624 communicates with the electronic device ED in a wireless (for example, Bluetooth, a wireless network, and an infrared ray) or wired (for example, a wired network or a cable) manner and provides the characteristic signal FS to the electronic device ED. An application program may be installed in the electronic device ED to record or analyze the characteristic signal FS. Therefore, the purpose of tracking or evaluating the blood vessel state can be achieved, which is not limited thereto.

The previous description of the present invention is provided to enable a person of ordinary skill in the art to make or implement the present invention. Various modifications to the present invention will be apparent to a person skilled in the art, and the general principles defined herein can be applied to other variations without departing from the spirit or scope of the present invention. Therefore, the present invention is not intended to be limited to the examples described herein, but is to be in accord with the widest scope consistent with the principles and novel features of the invention herein.

What is claimed is:

1. A vascular state measuring device, comprising:
a signal transceiver having a tunnel structure for arranging a to-be-measured part, wherein the signal transceiver is configured to output at least a first electromagnetic signal to the to-be-measured part toward the tunnel structure to generate an eddy current at the to-be-measured part, and receive a second electromagnetic signal generated by the corresponding eddy current;
a control module coupled with the signal transceiver, wherein the control module comprises:
a signal generation unit configured to generate an AC signal and provide the AC signal to the signal transceiver to generate the first electromagnetic signal; and
a processing unit configured to calculate a characteristic signal based on the first electromagnetic signal and the second electromagnetic signal, wherein the characteristic signal corresponds to at least one state of at least one blood vessel at the to-be-measured part.

2. The vascular state measuring device according to claim 1, wherein the to-be-measured part is a finger.

3. The vascular state measuring device according to claim 1, wherein the signal transceiver comprises:
a first coil configured to emit the first electromagnetic signal, wherein a hollow part of the first coil serves as a part of the tunnel structure.

4. The vascular state measuring device according to claim 3, wherein the signal transceiver further comprises a second coil;
wherein the first coil is arranged at a first position in the tunnel structure, the second coil is arranged at a second position in the tunnel structure, and a hollow part of the second coil serves as a part of the tunnel structure;
wherein a spacing is defined between the first position and the second position;
wherein a time difference exists between receiving of the second electromagnetic signal by the first coil and receiving of the second electromagnetic signal by the second coil; and
wherein the processing unit is configured to calculate the at least one state based on the spacing and the time difference.

5. The vascular state measuring device according to claim 4, wherein the at least one state comprises blood pressure of the at least one blood vessel at the to-be-measured part.

6. The vascular state measuring device according to claim 1, further comprising:
a matching element arranged on an inner wall of the tunnel structure, wherein a magnetic impedance of the matching element is between a magnetic impedance of the to-be-measured part and a magnetic impedance of the signal transceiver.

7. The vascular state measuring device according to claim 1, further comprising:
a depth detection unit configured to send a detection signal to the to-be-measured part and provide depth information corresponding to the at least one blood vessel to the control module, wherein the control module adjusts a frequency or a strength of the first electromagnetic signal based on the depth information.

8. The vascular state measuring device according to claim 1, wherein the signal transceiver further outputs at least one leading electromagnetic signal before outputting the first electromagnetic signal, wherein each of the at least one leading electromagnetic signal corresponds to a different signal parameter, and the signal parameter of the first electromagnetic signal corresponds to one of the at least one leading electromagnetic signal with an optimal response.

9. The vascular state measuring device according to claim 8, wherein the signal generation unit outputs at least one leading AC signal to generate the at least one leading electromagnetic signal, and each of the at least one leading electromagnetic signal corresponds to one of the at least one leading AC signal.

10. The vascular state measuring device according to claim 8, wherein the control module comprises an adjustable passive element coupled with the signal transceiver, and the control module adjusts the adjustable passive element to adjust a signal parameter of each of the at least one leading electromagnetic signal.

11. The vascular state measuring device according to claim 1, wherein the control module further comprises:
a communication unit coupled with the processing unit, wherein the communication unit is configured to output the characteristic signal to an electronic device.
